(19) United States
(12) Patent Application Publication
(10) Pub. No.: US 2006/0269571 A1
(43) Pub. Date: Nov. 30, 2006

(54) Equine West Nile virus immunotherapy

(76) Inventors: Roy Hall, Graceville (AU); Terry Kaleung Ng, Fort Dodge, IA (US); Hsien-Jue Chu, Fort Dodge, IA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/284,477

(22) Filed: Nov. 22, 2005

(30) Foreign Application Priority Data
Nov. 23, 2004 (AU) 2004906672

(51) Int. Cl. A61K 39/295

(52) U.S. Cl. 424/202.1

(57) ABSTRACT

An immunotherapeutic composition and prophylactic and/or therapeutic methods of treatment are provided for West Nile virus in animals, and particularly non-human animals, such as horses, wherein said first flavivirus is less virulent and/or pathogenic flavivirus than said second flavivirus The composition and method of treatment include a monoclonal antibody to Kunjin virus E protein, wherein the monoclonal antibody is capable of neutralizing West Nile virus in the non-human animal notwithstanding that West Nile virus is more virulent and/or pathogenic than Kunjin virus.

### CROSS-REFERENCE TO RELATED APPLICATIONS

This application claims the benefit of Australian Patent Application No. 2004906672, filed Nov. 23, 2004, which is hereby incorporated by reference in its entirety.

### FIELD OF THE INVENTION

This invention relates to immunotherapy of flaviviral infection in animals. More particularly, this invention relates to antibody-mediated immunotherapy of West Nile virus infection in equines, such as horses.

### DESCRIPTION OF THE RELATED ART

West Nile (WN) virus is a mosquito-transmitted flavivirus that produces a potentially fatal disease in human and non-human animals, such as horses, and has traditionally been associated with viral outbreaks in Europe and Africa (1). There have also been specific reports of outbreaks in Italy (2), South Africa (3), Israel (4), Mexico (5) and various states of the USA (6,7).

In 1999 WN virus appeared for the first time in the New World associated with an outbreak of a fatal or debilitating disease in humans and equines and extremely high levels of morbidity and mortality in several species of native birds in New York (8). Since its introduction into North America, WN virus has spread to most states of the USA and to parts of Canada and Mexico via mosquito-bird transmission cycles (9; cdc.gov/ncidod/dvbid/westnile/index.htm).

Kunjin (KUN) virus is a genetically stable Australian flavivirus which, although having 98 to 99% amino acid coding sequence homology with West Nile virus New York strain (10,11), produces only rare, non-fatal cases of human and equine disease (12), unlike the more virulent and pathogenic New York West Nile virus.

Indeed, comparative studies of the New York West Nile strain and KUN virus have revealed that 1000-10,000 fold more infectious virus of the latter is required to produce disease in adult mice by peripheral inoculation (12).

West Nile virus infection of both human and non-human animals is therefore a severe problem, particularly in non-human animals of commercial value, such as horses. The extremely virulent nature of West Nile virus and the aggressive disease pathology caused by even relatively low levels of virus has made immunotherapy of West Nile infection extremely difficult.

### SUMMARY OF THE INVENTION

The present invention therefore seeks to provide improved immunotherapy of West Nile virus infection of animals, inclusive of human and non-human animals

The invention is broadly directed to use of an antibody which binds a protein or protein fragment encoded by a less virulent and/or pathogenic flavivirus to passively immunize against at least one other more virulent and/or pathogenic flavivirus.

While the invention is directed to animals, inclusive of human and non-human animals, in a particularly preferred form the invention is directed to non-human animals, including but not limited to equines.

In a first aspect, the invention provides an immunotherapeutic composition comprising a monoclonal antibody which is capable of binding a protein or fragment thereof encoded by a first flavivirus, which monoclonal antibody is capable of neutralizing a second flavivirus upon administration of the monoclonal antibody to a non-human animal.

In a second aspect, the invention provides a method of therapeutically and/or prophylactically treating a flavivirus infection a non-human animal including the step of administering to said non-human animal a monoclonal antibody capable of binding a protein encoded by a first flavivirus to thereby prophylactically or therapeutically neutralize a second flavivirus that is, or is capable of, infecting said non-human animal.

In a preferred embodiment, said first flavivirus is Kunjin virus.

According to this embodiment, the monoclonal antibody is preferably an antibody which binds a Kunjin virus structural protein.

More preferably, the monoclonal antibody binds Kunjin virus E protein.

Preferably, the monoclonal antibody is selected from the group consisting of mAb 3.91D and mAb 3.67G.

A hybridoma that produces mAb 3.91D has been deposited at the American Type Culture Collection (ATCC), Manassas, Va. 20108 USA, on Oct. 28, 2004 with accession number PTA-6268.

A hybridoma that produces mAb 3.76G has been deposited at the American Type Culture Collection (ATCC), Manassas, Va. 20108, USA on Oct. 28, 2004 with accession number PTA-6267.

Even more preferably, the monoclonal antibody is mAb 3.91D as will be described in more detail hereinafter.

In another preferred embodiment, said second flavivirus is a strain of West Nile virus.

In a particularly preferred embodiment, said strain of West Nile virus is NY 99 strain.

In a third aspect, the invention provides a non-human animal treated according to the method of the second aspect.

In a fourth aspect, the invention provides a method of producing a biological product including the steps of obtaining said biological product from a non-human animal immunized with a monoclonal antibody capable of binding a protein encoded by a first flavivirus to thereby prophylactically or therapeutically neutralize a second flavivirus that is, or is capable of, infecting said non-human animal.

In a fifth aspect, the invention provides a biological product of a non-human animal produced according to the fourth aspect.

In particular embodiments the biological product may be a blood product such as immune or hyper-immune plasma, cells such as an immune cell or antigen-presenting cell isolated from said non-human animal.

Preferably, according to the aforementioned aspects, said first flavivirus is less virulent and/or pathogenic than said second flavivirus.

It will be appreciated that the invention provides immunotherapeutic compositions and methods for prophylactic or therapeutic treatment of animals, particularly non-human animals, which are susceptible to infection by a virulent and/or pathogenic flavivirus, particularly West Nile Virus strain NY 99.

It will therefore be appreciated that the invention is particularly applicable to non-human mammals such as equines, cows, sheep, pigs, dogs, cats, and the like, as well as other commercially important and/or domesticated species.

Furthermore, the invention is applicable to avians such as ducks, geese and chickens, for example.

Preferably, the non-human animal is an equine.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE DRAWING

FIG. 1. Results of mouse challenge experiments where mice received a lethal dose (100 infectious units) of WNV NY99 strain and were then administered either PBS at day 1 post infection, a single dose of 3.91D mAb at times ranging from day 1-6 post infection or control mAb (ME6) at day 1 post infection.

### DETAILED DESCRIPTION OF THE PREFERRED EMBODIMENT

The present invention, at least in part, arises from the unexpected finding that an antibody which binds Kunjin virus E protein, in particular the 3.91D monoclonal antibody (“mAb 3.91D”), is capable of neutralizing NY99 strain of West Nile virus, and that such an antibody is efficacious in the prophylactic and/or therapeutic treatment of West Nile virus infection in non-human animals such as horses.

This is surprising given that Kunjin virus is significantly less pathogenic than West Nile Virus NY99 strain in horses. Data from mouse experiments suggests that 1000-10,000 fold more infectious Kunjin virus (compared to West Nile virus) is required to produce symptomatic disease in these animals.

Thus mAb 3.91D displays surprising efficacy in terms of its ability to neutralize a lethal dose of West Nile virus at very low antibody concentrations.

In a particularly preferred embodiment, the invention therefore provides use of anti-Kunjin virus antibody such as mAb 3.91D or mAb 3.76G for therapeutic or prophylactic treatment of West Nile virus infection in non-human animals.

The term “nucleic acid” as used herein designates single-or double-stranded mRNA, RNA, and DNA inclusive of cDNA and genomic DNA.

By “protein” is meant an amino acid polymer. Amino acids may include natural (i.e genetically encoded), non-natural, D- and L-amino acids as are well known in the art.

A “peptide” is a protein having less than fifty (50) amino acids.

A “polypeptide” is a protein having fifty (50) or more amino acids.

As used herein, an “antibody” is an immunoglobulin protein or fragment thereof, that may comprise additional molecular components such as sugars, lipids and the like.

It will also be appreciated that an antibody may be modified to include a label such as biotin, a chromogen, a catalyst, an enzyme, a fluorophore, a chemiluminescent molecule, a radioisotope or a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes useful as labels is disclosed in U.S. Pat. No. 4,366,241, U.S. Pat. No. 4,843,000, and U.S. Pat. No. 4,849,338, all of which are herein incorporated by reference. Enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution.

By way of example, the fluorophore may be fluorescein isothiocyanate (FITC), oregon green, tetramethylrhodamine isothiocyanate (TRITL), allophycocyanin (APC), Cy3, Cy5 and R-Phycoerythrin (RPE), although without limitation thereto.

Typically, an antibody is an immunoglobulin produced by an animal or a cell isolated or at least partly derived therefrom, which immunoglobulin is capable of binding another molecule, usually referred to as an “antigen”.

In many cases, the antigen is, or is structurally similar to, at least a portion of the molecule administered to the animal to induce an antibody response thereto.

As will be understood by those of skill in the art, the immune response elicited by an animal in response to an antigen will usually be heterogeneous in the sense that multiple antibodies are typically produced, each with its own characteristic properties (e.g., in terms of heavy and light chain components, idiotype, antigen affinity and specificity) to bind the antigen. This heterogeneity may also be due to the presence of more than one epitope in the antigen.

Thus a “monoclonal antibody” is an antibody produced by a particular antibody-producing cell (e.g., of the B-lymphocyte/plasma cell lineage) or clonally-derived cell population, that produces a single, genetically encoded immunoglobulin having a particular and unique immunoglobulin structure and antigen-binding properties such as described above.

In typical practice, the antibody-producing cell is fused with a myeloma cell such as NS-1 or SP2, although without limitation thereto, to produce an immortalized hybridoma cell capable of essentially continuous growth and monoclonal antibody production.

A most preferred monoclonal antibody according to the present invention is mAb 3.91D. The production of this antibody and demonstration of the E-protein specificity of this antibody is described in reference 17.

As will be understood from the context of the present invention, “antibody” includes and encompasses any fragment, subunit or other modification thereof that retains the ability to neutralize West Nile virus upon administration to a non-human animal, preferably an equine such as a horse.

Thus, the invention contemplates use of heavy and/or light chain components, fragments such as Fab and F(ab′)2 fragments, isolated variable or hypervariable regions and/or complementarity determining region (CDR) domains alone or fused to another protein or peptide, although without limitation thereto.

Recombinant monoclonal antibodies, particularly recombinantly engineered or grafted antibodies or recombinant antibodies generated by phage display, are also contemplated.

It will be appreciated that the preferred antibody for use according to the present invention, mAb 3.91D and mAb 3.76G, are mouse monoclonal antibodies. Hence a particular antibody modification contemplated by the present invention is “grafting” a CDR domain (or other antigen-binding region) of a monoclonal antibody such as mAb3.91D onto an equine immunoglobulin “backbone” to thereby reduce the potential for an adverse immune response by a horse to the antibody once administered to the horse.

This principle may also be extended to “humanization” of antibodies and/or to antibodies for administration to non-human animals other than equines.

Grafting may be achieved by recombinant engineering or chemical synthetic means as are well known to persons skilled in the art.

Recombinant antibodies may be conveniently prepared with fusion partner sequences (such as glutathione-S-transferase, maltose binding protein or polyhistidine sequences) that assist affinity purification and, if desired, can be cleaved from the purified immunoglobulin by an appropriate protease.

General guidance regarding cloning, recombinant expression, and modification of antibody V regions may be found, for example, by reference to CURRENT PROTOCOLS IN IMMUNOLOGY Eds. Coligan et al. (John Wiley & Sons NY), particularly in Unit 2.12.

Reference is also made to Unit 17.1 of CURRENT PROTOCOLS IN IMMUNOLOGY, supra, which sets forth techniques generally applicable to bacteriophage library construction and selection of recombinant antibodies.

Monoclonal antibodies such as but not limited to mAb 3.91D and mAb 3.76G may be used according to the invention in a hybridoma cell culture supernatant, as a purified supernatant fraction, in ascites fluid (such as by growth of the hybridoma in a mouse peritoneum) or in substantially pure form.

A hybridoma that produces mAb 3.91D has been deposited at the American Type Culture Collection (ATCC) Manassas, Va. 20108 USA on Oct. 28, 2004 with accession number PTA-6268.

A hybridoma that produces mAb 3.76G has been deposited at the American Type Culture Collection (ATCC) Manassas, Va. 20108 USA on Oct. 28, 2004 with accession number PTA-6267.

Antibody purification is well known in the art and methods such as ammonium sulphate fractionation, protein A- or -G mediated purification, antigen-mediated affinity purification and cation exchange chromatography are readily applicable to the present invention.

More general guidance regarding antibody production and purification may be found, for example, by reference to CURRENT PROTOCOLS IN IMMUNOLOGY Eds. Coligan et al. (John Wiley & Sons NY), particularly in Units 2.4-2.10.

It is also noted that substantially purified mAb 3.91D is commercially available from Chemicon.

In particular embodiments, said monoclonal antibody is produced with a high Plaque-reduction neutralization test (PRNT) titer. Typically, although not exclusively, high PRNT titer monoclonal antibody production is effected by growth in roller bottles in the presence of serum free medium and chemically defined media.

PRNT titers in the range 15,000 to 30,000, or more particularly about 17,000 to 25,000, may be obtained by this procedure.

While it will be appreciated that the invention is applicable to any human or non-human animal, in a particularly preferred embodiment the invention is directed to immunotherapy of equines such as horses, although without limitation thereto.

As used herein, “equine” refers to any member of the genus Equus, which includes and encompasses Equus burchelli, the plains zebra of Africa, Equus zebra, the mountain zebra of South Africa, Equus grevyi, Grevy's zebra, Equus caballus, the true horse; Equus hemionus, the desert-adapted onager of Asia & the Mideast; and Equus asinus the true asses & donkeys of northern Africa.

In one broad form, the invention is directed to use of a monoclonal antibody which binds a protein or protein fragment encoded by a less virulent and/or pathogenic flavivirus to passively immunize against at least one other more virulent and/or pathogenic flavivirus.

As used herein, “flavivirus” and “flaviviral” refer to members of the family Flaviviridae within the genus Flavivirus, which contains 65 or more related viral species. Typically, flaviviruses are small, enveloped RNA viruses (diameter about 45 nm) with peplomers comprising a single glycoprotein E. Other structural proteins are designated C (core) and M (membrane-like). The single stranded RNA is infectious and typically has a molecular weight of about 4×10⁶ with an m7G ‘cap’ at the 5′ end but no poly(A) tract at the 3′ end; it functions as the sole messenger. Flaviviruses infect a wide range of vertebrates, and many are transmitted by arthropods such as ticks and mosquitoes, although a separate group of flaviviruses is designated as having no-known-vector (NKV).

Particular, non-limiting examples of flaviviruses are West Nile virus, Kunjin virus, Yellow Fever virus, Japanese Encephalitis virus, Dengue virus, Tick-borne encephalitis, Murray Valley encephalitis, Saint Louis encephalitis, Montana Myotis leukoencephalitis virus, Usutu virus, and Alkhurma virus.

In a preferred embodiment, the invention is directed to use of a monoclonal antibody which binds a Kunjin virus E protein, or a fragment thereof, to passively immunize against West Nile virus.

In this context, the term “fragment” refers to a sub-domain, region or peptide to which said monoclonal antibody is capable of binding. Such a fragment may therefore be, or comprise, an antigenic determinant or epitope which is required for antibody binding.

The epitope may be linear, discontinuous and/or conformational, as understood by persons skilled in the art.

Epitopes may be elucidated or “mapped”, with a view to determining the primary and/or conformational structure of the epitope bound by a particular antibody. Epitope mapping can be useful in designing epitopes for the production of improved antibodies that are potentially even more capable of neutralizing West Nile virus than is mAb 3.91D, for example.

General guidance with regard to epitope mapping may be found, for example, in Units 9.4 and 9.5 of CURRENT PROTOCOLS IN IMMUNOLOGY, supra.

More particularly, data obtained by the present inventors suggest that the 3.91D mAb binds to a neutralizing, conformational epitope comprising residue 332 of the West Nile virus E protein.

Preferably, residue 332 is a threonine residue or other residue that retains antigenicity (for example a serine or alanine).

Therefore, the invention contemplates use of any monoclonal antibody raised against Kunjin virus E protein which is capable of binding a West Nile virus E protein epitope that comprises residue 332 to thereby neutralize West Nile virus in a non-human animal.

In embodiments relating to said West Nile virus E protein conformational epitope, this may be a discontinuous epitope that further comprises at least one additional amino acid residue selected from the group consisting of Ser 306, Lys 307, and Thr 330.

In this regard, reference 20 provides enabling disclosure particularly relevant to epitope mapping of antibodies that bind West Nile virus E protein.

It is also contemplated that residues Asn 368 and Gln 391, which are on adjacent parts of the WN E protein, may also be part of the neutralizing epitope Immunotherapeutic compositions, vaccines and methods of immunization

In a particular aspect, the invention is broadly directed to an immunotherapeutic composition comprising a monoclonal antibody which binds a protein or protein fragment encoded by a less virulent and/or pathogenic flavivirus to prophylactically and/or therapeutically treat an infection by at least one other more virulent and/or pathogenic flavivirus.

A particular embodiment of the invention relates to use of a monoclonal antibody which binds a Kunjin virus-encoded protein to prophylactically or therapeutically treat West Nile virus NY99 strain in non-human animals.

According to one preferred embodiment of the present invention, it is proposed that administration of mAb 3.91D or 3.76G, for example, to a non-human animal provides “passive immunization” of the non-human animal.

By this is meant that the West Nile virus neutralizing ability of mAb 3.91D is transferred to the recipient non-human animal, to thereby immunize said non-human animal, without the recipient non-human animal necessarily eliciting its own immune response to West Nile virus.

The invention therefore contemplates administration of the monoclonal antibody prior to, concurrent with or following a West Nile virus infection.

Thus, in certain non-limiting embodiments the invention may be effective where, within a population (e.g., in a stable or zoo) one or more equines display symptoms of West Nile virus infection and/or are diagnosed with infection, while others are asymptomatic or harbour levels of West Nile virus infection below the level of diagnostic detection.

An immunization program could therefore be employed to prophylactically treat equines yet to display West Nile virus symptoms while also treating clearly infected horses.

In this regard, the present invention may be used alone or in conjunction with other West Nile virus immunization protocols such as described in reference 18, although without limitation thereto.

Typically, the monoclonal antibody is administered as an immunotherapeutic composition which may further comprise a pharmaceutically-acceptable carrier, diluent or excipient and, optionally, an adjuvant.

According to the invention, it will be understood that the pharmaceutically-acceptable carrier, diluent or excipient is suitable for use in a veterinary composition formulated according to the non-human animal recipient.

Suitably, said pharmaceutically-acceptable carrier, diluent or excipient is compatible with said monoclonal antibody and is immunologically acceptable to thereby enable effective administration of said monoclonal antibody to a non-human animal.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water. Diluents or excipients containing inorganic salts, amino acids, buffers, vitamins, antibiotics, and preservatives may also be used. Thus, the pharmaceutically-acceptable carrier, diluent or excipient may be aqueous, or non-aqueous, and may include oil-in-water and water-in-oil emulsions.

Any safe route of administration may be employed for providing a non-human animal with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Immunotherapeutic compositions of the present invention suitable for oral or parenteral administration may be presented as discrete units such as capsules, sachets or tablets each containing a pre-determined amount of monoclonal antibody, as a powder or granules or as a solution or a suspension in an aqueous liquid, a non-aqueous liquid, an oil-in-water emulsion or a water-in-oil liquid emulsion. Such compositions may be prepared by any of the methods of veterinary pharmacy.

Preferably, immunotherapeutic compositions of the invention are parentally administered to an equine animal as an aqueous solution, either intravenously or intramuscularly.

It will also be appreciated that immunotherapeutic compositions may, in certain embodiments, include an adjuvant.

As will be understood in the art, an “adjuvant” means one or more substances that enhances the immunogenicity and/or efficacy of an immunotherapeutic composition. Non limiting examples of suitable adjuvants include squalane and squalene (or other oils of animal origin); block copolymers; detergents such as Tween® 80; Quil® A, mineral oils such as Drakeol or Marcol, vegetable oils such as peanut oil; Corynebacterium derived adjuvants such as Corynebacterium parvum; Propionibacterium derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin aluminium hydroxide or Quil A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE Dextran or with aluminium phosphate; carboxypolymethylene such as Carbopol′ EMA; acrylic copolymer emulsions such as Neocryl A640 (e.g., U.S. Pat. No. 5,047,238); vaccinia or animal poxvirus proteins; sub viral particle adjuvants such as cholera toxin, or mixtures thereof.

The above compositions may be administered in a manner compatible with the dosage formulation, and in such amount as is effective. The dose administered to a non-human animal, in the context of the present invention, should be sufficient to effect a beneficial response in the non-human animal over an appropriate period of time. The quantity of agent(s) to be administered and the frequency of administration, may depend on the non-human animal to be treated inclusive of the age, sex, weight and general health condition thereof, factors that will depend on the judgment of the veterinary practitioner.

In a preferred embodiment, the monoclonal antibody is administered at a concentration of 0.1-5.0 mg/mL, or more preferably at a concentration of 0.5-1.0 mg/mL.

As hereinbefore described, PRNT titers of mAb 3.91D in the range 15,000 to 30,000, or more particularly about 17,000 to 25,000, have been obtained according to the present invention.

These preparations contained approximately 0.74 mg/mL of IgG3 as measured by ELISA.

With this in mind, in a particular non-limiting embodiment, a suitable dosage of monoclonal antibody for parenteral administration (such as intravenous and/or intramuscular injection of an aqueous solution comprising said monoclonal antibody), may be in the range 1 mL to 100 mL (e.g., 0.74 mg to 74.0 mg) per 100 kg body weight. This includes dosages in the range 5 mL to 50 mL (e.g., 3.7 mg to 37.0 mg) per 100 kg body weight, 7 mL to 30 mL (e.g., 5.18 mg to 22.2 mg) per 100 kg body weight and any sub-ranges within the aforementioned ranges together with ranges between any of those stated above. Non limiting examples of such sub-ranges include 1 mL to 5 mL (e.g., 0.74 mg to 3.7 mg) per 100 kg body weight and 50 mL to 100 mL (e.g., 37.0 mg to 74.0 mg) per 100 kg body weight).

As will be apparent from the Examples, administration to horses of high PRNT titer mAb 3.91D at 7.4 (5.48 mg), 14.8 (10.95 mg) and 29.6 mL (21.9 mg)/100 kg have been specifically exemplified.

In light of the foregoing, it will be apparent to the skilled person that the invention has been exemplified herein using high PRNT titer mAb3.91G. Accordingly, administered volumes will be readily varied according to the titer and/or the particular monoclonal antibody to be administered.

The present invention also contemplates biological products or materials isolated from West Nile virus-infected animals, preferably horses, treated or passively immunized according to the present invention. Such biological products or materials may include blood products inclusive of serum, serum antibodies, plasma or hyper-immune plasma, complement, isolated cells such as dendritic cells, macrophages and other antigen-presenting cells, T and/or B lymphocytes, NK cells or any other cells that are involved in the initiation, regulation and/or maintenance of cell mediated immunity. Isolated cells may have efficacy in dendritic cell therapy, production of monoclonal antibodies and adoptive transfer, for example, although without limitation thereto.

So that the invention may be readily understood and put into practical effect, the skilled person is directed the following non-limiting examples.

### EXAMPLES

### Materials and Methods

Cell culture and Virus Preparations

Vero cells were grown in HEPES-buffered Medium 199 (Gibco) supplemented with antibiotics and 10% FBS and incubated at 37° C. C6/36 cells were cultured in Medium 199 supplemented with antibiotics and 10% FBS and incubated at 28° C. and 5% CO₂. For virus stock production, Vero cells were infected with KUN virus (MRM61C strain; 4), WN virus (NY99-4132 strain, obtained from the Division of Vector-Borne Infectious Diseases, Centers for Disease Control, Fort Collins), or FLSD virus (derived from KUN cDNA clone FLSD; 13, 14) at a multiplicity of infection of 0.1-1 and cultured in medium supplemented with 2% FBS. Culture supernatant was harvested and clarified at 72-96 h post-infection when 50-70% of cells showed cytopathic effects (CPE). The concentration of infectious virus in stocks was determined by titration on Vero cells in 96-well plates and calculated as the 50% infectious dose (ID₅₀)/ml (13). One ID₅₀ is equivalent to one infectious unit (i.u.).

Production of Mouse Antiserum.

Mice were immunized via the intra-peritoneal (i.p.) route with 10³ i.u. of the KUN virus (FLSD strain) and bled for serum 19 days later. Mouse blood was collected by tail bleed, held at 4° C. overnight to clot, and serum separated and snap frozen at −70° C. until tested.

Production of Monoclonal Antibodies

Monoclonal antibodies 3.91D and 3.67G reactive to the E protein of KUN virus (16) were produced as sterile, cell-free, high-titre hybridoma culture supernatants using serum-free medium and stored at 4° C.

Monoclonal antibodies were also produced in large quantities in roller bottles and bioreactors. Cultures were incubated at 37° C. for 12 to 20 days in 850 cm² roller bottles containing media up to 500 mL of media and 15 liter Applikon (Forest City, Calif.) bioreactors containing 10 liters of media. Cultures prepared in serum free media such as, chemically defined CD Hybridoma medium and Serum Free Hybridoma medium (LTI, Grand Island, N.Y.) supplemented with gentamycin and L-glutamine. Stocks of culture harvest were processed using a 0.45 μm hollow fiber cartridge to remove cell debris. The permeates collected were pooled and stored at 4° C.

Measurement of Serum and Monoclonal Antibody Reaction to KUN and WN Antigens in ELISA

Sera from each mouse group were pooled and titrated in doubling dilutions in fixed-cell ELISA against the viral proteins of wild type KUN virus and NY99 WN virus as previously described (15). Hybridoma supernatants containing mAbs 3.91D or 3.67G were similarly diluted and tested. The reciprocal of the serum or mAb dilution that produced an OD of at least 0.3 on viral antigen and at least 0.2 higher than that produced on control antigen (fixed uninfected cells) was deemed the ELISA titre of each sample.

Microneutralization Assays

Sera or mAb samples were tested for neutralization of KUN and WN viruses by microneutralisation assay as described previously (16). Briefly, pooled sera from each mouse group or undiluted hybridoma supernatant, were heat-inactivated at 56° C. and serially diluted two-fold in cell growth medium. Twenty-five μl of each dilution was then added in duplicate to wells of a 96 well culture plate. An equal volume of growth medium containing approximately 100 i.u. of virus was then added to each well, and plates were allowed to incubate for one hour at 37° C. with occasional gentle agitation. Fifty μl of growth media containing approximately 10⁴ Vero cells were then added to each well and plates incubated at 37° C. for 5 days. The reciprocal of the serum dilution that inhibited the formation of viral cytopathic effects was deemed the neutralization titre.

Mouse Challenge with West Nile Virus NY99 Strain

Eight week-old female Balb/c mice were challenged with 100 infectious units of WNV NY99 strain by intraperitoneal (i.p.) injection of a 100 μL inoculum. Groups of 20 mice were administered a single dose of 50 μg of mAb 3.91D in PBS by i.p. injection at times ranging from days 1-6 post infection. One group received 50 μg of MAb ME6, which is specific for Murray Valley encephalitis virus NS1 protein, at day 1 post infection. Additionally, another group received 100 μL of PBS by i.p. injection at day 1 post infection to determine the mortality rate in mock-treated mice. Mice were inspected for symptoms of neurological disease for 21 days.

Plaque Reduction Neutralization Test (PRNT)

Samples of horse serum or monoclonal antibody preparation were tested for WNV antibody titer using PRNT. Serum or monoclonal antibody samples were tested using Vero cells at a 90% plaque-reduction level against WNV (strain VM-2 received from Dr. E. Ostlund of the NVSL, Ames, Iowa). Briefly, a two-fold serial dilutions of serum samples were allowed to incubate overnight at 4° C. with reference virus of WNV at 200 PFU/0.1 mL. The virus-serum mixture were then transferred to Vero cell monolayers, overlayed and allowed to incubate at 37° C. (±2° C.) for 48-60 hours. The wells were then stained for 24-48 hours with neutral red overlay and plaques were counted using a light box.

Pharmacokinetics of Immunotherapy in Horses

Thirty (30) West Nile virus seronegative horses were treated intravenously with a preparation of monoclonal antibody 3.91D. The potency of the preparation was measured to have a PRNT antibody titer of 17,963. Horses were randomized into four study groups. The first ten horses were assigned to Group 1, the next ten horses to Group 2 and the following five horses to Group 3. The remaining five horses were assigned to Group 4. The horses were weighed prior to treatment. Group 1, 2 and 3 horses were injected with 7.4, 14.8 and 29.6 mL/100 Kg of body weight of the preparation respectively. The appropriate amount of CD Medium was used to prepared the lower dose preparations. The Group 4 animals, injected with 14.8 mL/100 Kg of CD medium also served as environmental controls.

Individual horses were weighed prior to the treatment. Horses in Groups 1 to 3 were treated with a single dose regimen by administration in the neck area with an intraveneous undiluted dose of Mab 3.91D preparation. Group 4 horses were administered CD Hybridoma medium (LTI, Grand Island, N.Y.) supplemented with gentamycin and L-glutamine as injection site controls and as controls for detection of WNV exposure.

Pharmacokinetic Evaluation of Immunotherapy in Horses

The kinetics of decay of West Nile virus antibody in horses after intravenous injection using preparations outlined in Table 1 was evaluated by the functional assay of PRNT daily for 14 days.

### Results

Serum from KUN-immunised mice reacted with KUN and WN virus antigens in ELISA with similar efficiency with titres ranging from 640 to 1280. Both 3.91D and 3.67G mAbs showed high levels of reaction to both virus in ELISA with titres ranging from 2560-5120 for both viruses.

KUN-immune mouse serum also neutralized KUN and WN viruses with similar efficiencies with moderate titers ranging from 40-80. This correlates with observations that KUN-immunized mice were also protected from lethal challenge with WN virus (19). However, the most impressive results were obtained with Mabs 3.91D and 3.67G, which neutralized both viruses even at very high dilutions of hybridoma supernatant (titres ranged from 1280-5120 as listed in Table 2). These results demonstrate that 3.91D and 3.67G mAb bind with high affinity to critical neutralisation epitopes conserved on the E protein of both KUN and WN viruses.

Data obtained by the present inventors suggest that the 3.91D mAb binds to a conformational epitope comprising threonine residue 332 of the WNV E protein. This may be a discontinuous epitope that further comprises at least one additional amino acid residue selected from the group consisting of Ser 306, Lys 307, and Thr 330.

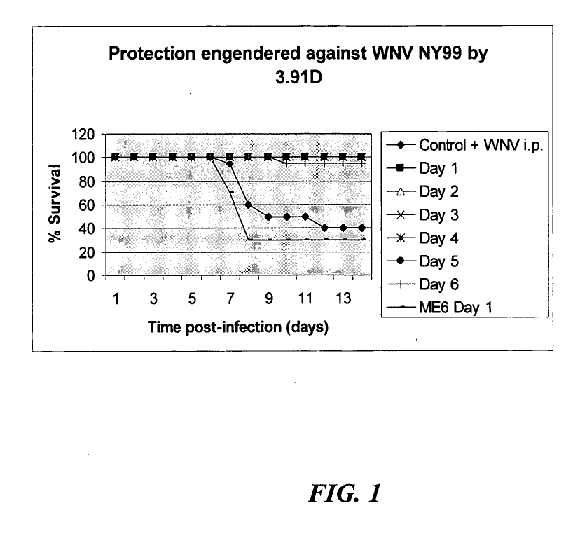

Virus challenge experiments were undertaken, the results of which are shown in FIG. 1. Most mice challenged with a normally lethal does of WNV NY99 strain survived for the observed period, even in cases where the 3.91D mAb was administered as late as 5 or 6 days post-infection. Mice receiving PBS or ME6 mAb displayed about 50% and 70% mortality respectively.

MAb 3.91D supernatant produced in large scale in roller bottles and biorectors using serum free and chemically defined media and measured functionally by PRNT against West Nile virus New York 99 strain (VM-2) had extraordinarily high titre of neutralizing activity. The WNV PRNT antibody titre of such preparations ranged from 17,000 to 24,000.

The pharmacokinetic persistency of the monoclonal antibody level in the serum samples with Mab 3.91D preparations injected into horses intraveneously appeared to be dose dependent. The antibody level as measured by PRNT increased from a dosage of 7.4 mL/100 Kg to 29.6 mL/100 Kg. Four of ten horses in the 7.4 mL/100 Kg dose had a detectable antibody of 5 on day 3 post administration whereas two of five horses had a detectable antibody of 5 (see Table 3) on day 9 after the injection in the group of horses administered with the dosage of 29.6 mL/100 Kg. No West Nile antibody was detected in horses injected with the growth medium (LTI CD Medium). No anaphylaxis or other adverse reactions were observed. Overall, considerable efficacy of the invention was demonstrated by these test results.

In light of the foregoing it will be appreciated that in a preferred form the invention provides immunotherapy of West Nile virus in non-human animals such as horses and other equines, and other mammals, which is effected by administration of anti-Kunjin virus E protein antibodies which surprisingly neutralize West Nile virus in the non-human animal.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

1. Murgue et al., (2002) Curr. Top. Microbiol. Immunol. 267 195.
2. Romi et al., (2004) Med. Vet. Entomol. 18 14.
3. Guthrie et al., (2003) Equine Vet. 35 601.
4. Banet-Noach et al., (2003) Virus Genes 26 135.
5. Ulloa et al., (2003) Vector Borne Zoonotic Dis. 3 3.
6. J. Am. Vet. Med. Assoc. (2003) 222 1669.
7. Zeller & Schuffenecker (2004) Eur. J. Clin. Microbiol. Infect. Dis. 27 209.
8. Lanciotti et al.,. (1999). Science 286 2333.
9. Roehrig et al., (2002). Curr. Top. Microbiol. Immunol. 267 223.
10. Coia et al., (1988). J. Gen. Virol. 69 1.
11. Lanciotti et al., (2002) Virology 298 96.
12. Hall et al., (2002). Curr. Top. Microbiol. Immunol. 267 253.
13. Hall et al., (1999). Virology, 264 66.
14. Khromykh et al., (1998). J. Virol. 72 7270.
15. Hall et al., (1996) J. Gen. Virol. 77 1287.
16. Hall et al., (1990). J. Gen. Virol. 71 2923.
17. Adams et al., (1995) Virology 206 49.
18. Ng et al., (2003) Dev. Biol. 114 221.
19. Hall et al., (2003) PNAS 100 10460.

20. Nybakken et al., (2005) Nature 437 764.

TABLE 1
Experimental design of Immunotherapy in horses

| | | Potency of | | | |
|---|---|---|---|---|---|
| | Prepa- | preparation | | | No. of |
| Group | ration | (PRNT) | Route | Dose | horses |
| 1 | Mab 3.91D | 17,963 | IV | 7.4 mL/ | 10 |
| | | | | 100 Kg | |
| 2 | Mab 3.91D | 17,963 | IV | 14.8 mL/ | 10 |
| | | | | 100 Kg | |
| 3 | Mab 3.91D | 17,963 | IV | 29.6 mL/ | 5 |
| | | | | 100 Kg | |
| 4 | LTI CD | NA | IV | 14.8 mL/ | 5 |
| (Control) | medium | | | 100 Kg | |

TABLE 2
Reaction of KUN-immune mouse sera and anti-KUN Mabs in
ELISA and virus neutralization assays (expressed as reciprocal
of highest dilution to inhibit CPE by virus).

| | |
|---|---|
| Neutralization | ELISA |

| Serum/Mab | KUN | WN | KUN | WN |
|---|---|---|---|---|
| Control# | <10 | <10 | <40 | <40 |
| anti- KUN## | 40 | 80 | 1280 | 640 |
| Mab 3.67G* | 1280 | 2560 | 2560 | 2560 |
| Mab 3.91D* | 5120 | 5120 | 5120 | 5120 |

#sera from unimmunised mice
##Sera from mice immunised with KUN virus (FLSD) (and subsequently protected from West Nile virus challenge)
*culture supernatants from hybridoma lines were tested in two-fold dilutions

TABLE 3
Pharmacokinetics of West Nile Virus PRNT antibody titer post treatment of immunotherapy in horses

| Horse | Treatment | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| ID | Group* | DPA | DPA | DPA | DPA | DPA | DPA | DPA | DPA | DPA | DPA | DPA | DPA | DPA | DPA | DPA | DPA |
| 3 | 1 | 10 | <5 | <5 | <5 | <5 | <5 | <5 | <5 | <5 | <5 | <5 | <5 | <5 | <5 | <5 | <5 |
| 4 | 1 | 5 | 5 | <5 | <5 | <5 | <5 | <5 | <5 | <5 | <5 | <5 | <5 | <5 | <5 | <5 | <5 |
| 14 | 1 | 5 | <5 | <5 | <5 | <5 | <5 | <5 | <5 | <5 | <5 | <5 | <5 | <5 | <5 | <5 | <5 |
| 16 | 1 | 10 | 5 | <5 | <5 | <5 | <5 | <5 | <5 | <5 | <5 | <5 | <5 | <5 | <5 | <5 | <5 |
| 17 | 1 | 20 | 5 | 5 | <5 | <5 | <5 | <5 | <5 | <5 | <5 | <5 | <5 | <5 | <5 | <5 | <5 |
| 23 | 1 | 10 | 5 | <5 | <5 | <5 | <5 | <5 | <5 | <5 | <5 | <5 | <5 | <5 | <5 | <5 | <5 |
| 27 | 1 | 10 | 5 | 5 | <5 | <5 | <5 | <5 | <5 | <5 | <5 | <5 | <5 | <5 | <5 | <5 | <5 |
| 32 | 1 | 10 | 5 | 5 | <5 | <5 | <5 | <5 | <5 | <5 | <5 | <5 | <5 | <5 | <5 | <5 | <5 |
| 33 | 1 | 5 | 5 | <5 | <5 | <5 | <5 | <5 | <5 | <5 | <5 | <5 | <5 | <5 | <5 | <5 | <5 |
| 35 | 1 | 5 | 10 | 5 | <5 | <5 | <5 | <5 | <5 | <5 | <5 | <5 | <5 | <5 | <5 | <5 | <5 |
| | GMT | 8.1 | 4.7 | 3.3 | 2.5 | 2.5 | 2.5 | 2.5 | 2.5 | 2.5 | 2.5 | 2.5 | 2.5 | 2.5 | 2.5 | 2.5 | 2.5 |
| 2 | 2 | 20 | 10 | 10 | 5 | 5 | <5 | <5 | <5 | <5 | <5 | <5 | <5 | <5 | <5 | <5 | <5 |
| 6 | 2 | 20 | 10 | ≧20 | 5 | 5 | 5 | <5 | <5 | <5 | <5 | <5 | <5 | <5 | <5 | <5 | <5 |
| 8 | 2 | 20 | 10 | 5 | 5 | 5 | 5 | <5 | <5 | <5 | <5 | <5 | <5 | <5 | <5 | <5 | <5 |
| 10 | 2 | 20 | 10 | 10 | 5 | 5 | 5 | <5 | <5 | <5 | <5 | <5 | <5 | <5 | <5 | <5 | <5 |
| 15 | 2 | 20 | 5 | 5 | 5 | 5 | <5 | <5 | <5 | <5 | <5 | <5 | <5 | <5 | <5 | <5 | <5 |
| 20 | 2 | 20 | 10 | ≧20 | 5 | 5 | 5 | <5 | <5 | <5 | <5 | <5 | <5 | <5 | <5 | <5 | <5 |
| 29 | 2 | 10 | 10 | ≧20 | 5 | 10 | 5 | <5 | <5 | <5 | <5 | <5 | <5 | <5 | <5 | <5 | <5 |
| 30 | 2 | 10 | 10 | 10 | 10 | 10 | 10 | 10 | 5 | <5 | <5 | <5 | <5 | <5 | <5 | <5 | <5 |
| 31 | 2 | 40 | 10 | 10 | 10 | 10 | 10 | 5 | 5 | <5 | <5 | <5 | <5 | <5 | <5 | <5 | <5 |
| 34 | 2 | 10 | 10 | 10 | 10 | 5 | 5 | <5 | <5 | <5 | <5 | <5 | <5 | <5 | <5 | <5 | <5 |
| | GMT | 17.4 | 9.3 | 10.7 | 6.2 | 6.2 | 5.0 | 3.1 | 2.9 | 2.5 | 2.5 | 2.5 | 2.5 | 2.5 | 2.5 | 2.5 | 2.5 |
| 12 | 3 | 20 | ≧20 | 10 | 10 | 10 | 10 | 5 | <5 | 5 | <5 | <5 | <5 | <5 | <5 | <5 | <5 |
| 19 | 3 | 40 | ≧20 | ≧20 | 10 | 10 | 10 | 5 | <5 | <5 | <5 | <5 | <5 | <5 | <5 | <5 | <5 |
| 21 | 3 | 40 | ≧20 | 10 | 10 | 20 | 10 | 5 | 5 | <5 | <5 | <5 | <5 | <5 | <5 | <5 | <5 |
| 22 | 3 | 40 | 10 | 10 | 10 | 10 | 5 | 5 | <5 | 5 | <5 | <5 | <5 | <5 | <5 | <5 | <5 |
| 25 | 3 | 40 | ≧20 | 5 | 5 | 10 | 10 | 10 | 5 | <5 | <5 | <5 | <5 | <5 | <5 | <5 | <5 |
| | GMT | 34.8 | 17.4 | 10.0 | 8.7 | 11.5 | 8.7 | 5.7 | 3.3 | 3.3 | 2.5 | 2.5 | 2.5 | 2.5 | 2.5 | 2.5 | 2.5 |
| 1 | 4 | <5 | <5 | <5 | <5 | <5 | <5 | <5 | <5 | <5 | <5 | <5 | <5 | <5 | <5 | <5 | <5 |
| 5 | 4 | <5 | <5 | <5 | <5 | <5 | <5 | <5 | <5 | <5 | <5 | <5 | <5 | <5 | <5 | <5 | <5 |
| 11 | 4 | <5 | <5 | <5 | <5 | <5 | <5 | <5 | <5 | <5 | <5 | <5 | <5 | <5 | <5 | <5 | <5 |
| 18 | 4 | <5 | <5 | <5 | <5 | <5 | <5 | <5 | <5 | <5 | <5 | <5 | <5 | <5 | <5 | <5 | <5 |
| 26 | 4 | <5 | <5 | <5 | <5 | <5 | <5 | <5 | <5 | <5 | <5 | <5 | <5 | <5 | <5 | <5 | <5 |
| | GMT | 2.5 | 2.5 | 2.5 | 2.5 | 2.5 | 2.5 | 2.5 | 2.5 | 2.5 | 2.5 | 2.5 | 2.5 | 2.5 | 2.5 | 2.5 | 2.5 |

GMT = Geometric mean titre
DPA = Days post administration
*See Table 1 for experimental design and treatment dosages

What is claimed is:

1. An immunotherapeutic composition comprising a monoclonal antibody which is capable of binding a protein or fragment thereof encoded by a first flavivirus, which monoclonal antibody is capable of neutralizing a second flavivirus upon administration of the monoclonal antibody to a non-human animal, wherein said first flavivirus is less virulent and/or pathogenic flavivirus than said second flavivirus.

2. The immunotherapeutic composition of claim 1, wherein the first flavivirus is Kunjin virus and the second flavivirus is West Nile virus.

3. The immunotherapeutic composition of claim 2, wherein the monoclonal antibody binds Kunjin virus E protein.

4. The immunotherapeutic composition of claim 4 wherein the monoclonal antibody is capable of binding a conformational epitope of West Nile virus E protein.

5. The immunotherapeutic composition of claim 4, wherein the West Nile virus E protein epitope comprises threonine residue 332.

6. The immunotherapeutic composition of claim 5, wherein the West Nile virus E protein epitope further comprises an amino acid residue selected from the group consisting of Ser 306, Lys 307 and Thr 330.

7. The immunotherapeutic composition of claim 6, wherein the West Nile virus E protein epitope further comprises an amino acid residue selected from the group consisting of Asn 368 and Gln 391.

8. The immunotherapeutic composition of claim 3, wherein the monoclonal antibody is selected from the group consisting of mAb 3.91D and mAb 3.67G.

9. The immunotherapeutic composition of claim 1 which is formulated for intravenous or intramuscular injection.

10. The immunotherapeutic composition of claim 1 which is formulated for administration to an equine.

11. The immunotherapeutic composition of claim 1 wherein the monoclonal antibody is at a plaque-reduction neutralization test (PRNT) titer of 15,000-30,000.

12. The immunotherapeutic composition of claim 1 wherein the monoclonal antibody is at a PRNT titer of 17,000-25,000.

13. The immunotherapeutic composition of claim 1 wherein the monoclonal antibody is at a concentration of about 0.5 to 1.0 mg/mL.

14. An immunotherapeutic composition for treating a West Nile virus infection of an equine, said composition comprising a monoclonal antibody selected from the group consisting of mAb 3.91D and mAb 3.67G at a dosage sufficient to neutralize said West Nile virus.

15. The immunotherapeutic composition of claim 14, wherein the dosage is 5-50 mL/100 kg body weight of said monoclonal antibody at a PRNT titer of 15,000-30,000 or a concentration of 0.5 to 1.0 mg/mL.

16. A method of therapeutically and/or prophylactically treating a flavivirus infection in a non-human animal including the step of administering to said non-human animal a monoclonal antibody capable of binding a protein encoded by a first flavivirus to thereby prophylactically or therapeutically neutralize a second flavivirus that is, or is capable of, infecting said non-human animal, wherein said first flavivirus is less virulent and/or pathogenic flavivirus than said second flavivirus.

17. The method of claim 16, wherein the first flavivirus is Kunjin virus and the second flavivirus is West Nile virus.

18. The method of claim 17, wherein the monoclonal antibody binds Kunjin virus E protein.

19. The method of claim 18 wherein the monoclonal antibody is capable of binding a conformational epitope of West Nile virus E protein.

20. The method of claim 19, wherein the West Nile virus E protein epitope comprises threonine residue 332.

21. The method of claim 20, wherein the West Nile virus E protein epitope further comprises an amino acid residue selected from the group consisting of Ser 306, Lys 307, and Thr 330.

22. The method of claim 21, wherein the West Nile virus E protein epitope further comprises an amino acid residue selected from the group consisting of Asn 368 and Gln 391.

23. The method of claim 18, wherein the monoclonal antibody is selected from the group consisting of mAb 3.91D and mAb 3.67G.

24. The method of claim 16, wherein the monoclonal antibody is administered intravenously or intramuscularly.

25. The method of claim 16, wherein the non-human animal is an equine.

26. The method of claim 16, wherein the non-human animal is passively immunized against West Nile virus infection.

27. The method of claim 15, wherein the monoclonal antibody is at a PRNT titer of 15,000-30,000.

28. The method of claim 25, wherein the monoclonal antibody is at a PRNT titer of 17,000-25,000.

29. The method of claim 15, wherein the monoclonal antibody is at a concentration of 0.5-1.0 mg/mL.

30. The method of claim 15, wherein the monoclonal antibody is administered at a dosage in the range 5-50 mL/100 kg body weight.

31. A method of therapeutically and/or prophylactically treating a West Nile virus infection in an equine including the step of administering to said equine a monoclonal antibody selected from the group consisting of mAb 3.91D and mAb 3.67G at a dosage sufficient to neutralize said West Nile virus.

32. The method of claim 31, wherein the dosage is 5-50 mL/100 kg body weight of said monoclonal antibody at a PRNT titer of 15,000-30,000 or at a concentration of 0.5-1.0 mg/mL.

33. A method of producing a biological product including the steps of obtaining said biological product from a non-human animal immunized with a monoclonal antibody capable of binding a protein encoded by a first flavivirus to thereby prophylactically or therapeutically neutralize a second flavivirus that is, or is capable of, infecting said non-human animal, wherein said first flavivirus is less virulent and/or pathogenic flavivirus than said second flavivirus

34. The method of claim 33, wherein the biological product is a blood product.

35. The method of claim 34, wherein the blood product is immune or hyper-immune plasma, serum, serum antibody, a lymphocyte or an antigen-presenting cell.

36. The method of claim 33, wherein the monoclonal antibody is administered intravenously or intramuscularly.

37. The method of claim 33, wherein the non-human animal is an equine.
